(19) United States
(12) Patent Application Publication
(10) Pub. No.: US 2004/0171560 A1
(43) Pub. Date: Sep. 2, 2004

(54) Stabilized pharmaceutical composition

(75) Inventors: Rama Mukherjee, Uttar Pradesh (IN); Arnand C. Burman, Uttar Pradesh (IN); Dhiraj Khattar, Uttar Pradesh (IN); Mukesh Kumar, Uttar Pradesh (IN)

Correspondence Address: NIXON & VANDERHYE, PC, 1100 N GLEBE ROAD, 8TH FLOOR, ARLINGTON, VA, 22201-4714, US

(73) Assignee: DABUR RESEARCH FOUNDATION, Ghaziabad (IN)

(21) Appl. No.: 10/739,405

(22) Filed: Dec. 19, 2003

Related U.S. Application Data
(60) Provisional application No. 60/435,353, filed on Dec. 23, 2002.

(51) Int. Cl. A61K 31/7048; A61K 31/337; A61K 31/4745; A61K 9/20

(52) U.S. Cl. 514/27; 424/465; 514/283; 514/449

(57) ABSTRACT

A stabilized pharmaceutical composition of anticancer drug and a solvent like dehydrated alcohol is disclosed. A method is disclosed that includes the addition of a stabilizing agent Malic acid having a unique triple action property of being an antioxidant, a chelating agent and an acidifying agent. Compositions prepared using this pre-treated dehydrated alcohol enhance the stability of paclitaxel in alcohol. A method of removing ionic, metallic and oxidizing impurities from alcohol using ion exchange and clay treatment is also disclosed.

### FIELD OF THE INVENTION

The present invention relates to a stabilized pharmaceutical composition in a solvent like dehydrated alcohol. More particularly, the present invention relates to stabilized nanoparticle formulation of anti cancer drugs presented in a kit form suitable for bedside reconstitution.

### BACKGROUND OF THE INVENTION

Administration of pharmaceutical compounds, particularly by injection, usually requires a suitable solvent or delivery system to enable the composition to be administered to a patient.

An ideal solvent must typically have the following properties:

it must be capable of solubilizing therapeutically effective amount of the active agent to produce an effective composition.

1. it must be compatible with the active agent

2. it should be safe i.e. it should not cause any toxicity to the patient.

it should produce a composition having a good shelf life.

Many solvents while possessing most of the above advantageous qualities are not particularly efficient in solubilizing the pharmaceutical agent to produce a stable composition for administration.

A potential problem associated with such solvents is that acids, salts or other ionic impurities, as well as residual water in the solvent, even if within the acceptable limits, can catalyze the degradation of the pharmaceutical agent. A solvent with sufficiently low levels of particularly deleterious impurities will yield a more stable pharmaceutical agent containing compositions.

In particular, pharmaceutical composition of Taxol the FDA approved composition of Paclitaxel marketed by Bristol Myers Squibb in a co-solvent of 50:50 by volume of dehydrated ethanol and commercial grade Cremophor EL exhibit a loss of potency of greater than 60% after storage for 12 weeks at 50.degree C. (U.S. Pat. No. 5,504,102). The loss of potency is attributed to the decomposition of paclitaxel during storage. It is believed that carboxylate anions present in Cremophor EL catalyze the decomposition of paclitaxel, even at levels within the defined limits set forth in the National Formulary.

A number of patents, briefly outlined below, disclose methods to overcome this stability issue of paclitaxel in the approved FDA formulation by improving the quality of cremophor.

Agharkar et al in U.S. Pat. No. 5,504,102 disclose removing the carboxylate anions from polyethoxylated castor oils (cremophor) by acid addition or alumina adsorption leading to a stabilized formulation.

Nikolayev et al in U.S. Pat. No. 5,925,776 disclose a method of reducing the cation content in the polyethoxylated castor oil (cremophor). This is achieved by pre-treating the polyethoxylated castor oil with a strong cation exchange resin. The stability of paclitaxel formulated in a mixture of low cationic content polyethoxylated castor oil of the invention and ethyl alcohol is shown to be better as compared to a formulation using untreated polyethoxylated castor oil of the invention and ethyl alcohol.

Anevski et al in U.S. Pat. No. 6,388,112 disclose a process for purifying a non-ionic surfactant or solvent capable of dispersing and solubilizing a pharmaceutical compound. In the process, a solution of solvent and alcohol is brought in contact with an activated carbon column and an ion exchange resin column. The process is particularly adapted to the purification of polyethoxylated castor oils. The purified solvent is useful in the preparation of pharmaceutical compositions having enhanced shelf life, such as for use with paclitaxel.

Carver et al in U.S. Pat. No. 6,306,894, (also U.S. Pat. Nos.6,140,359, 5,977,164, 5,972,992 and 5,733,888) disclose a pharmaceutical formulation of paclitaxel and polyethoxylated castor oil wherein the formulation is relatively acidified to a pH of less than 8.1 and preferably within a pH range of 5 to 7, inclusively. Ethanol is optionally included in the formulation. A variety of acidifying agents, a preferred one being anhydrous citric acid, are described.

Owens et al in U.S. Pat. No. 6,071,952 disclose a pharmaceutical composition with long-term storage stability containing a taxane or taxoid, a solubilizing/dispersing agent and an effective amount of an antioxidant.

Dralle-Voss et al in U.S. Pat. No. 6,096,911 disclose a process for purifying alkaoxylated fats (especially ethoxylated castor oil like cremophor EL) for the production of a stabilized pharmaceutical formulation by treatment with a solid substance, wherein a mixture of aluminium oxide and a silicate is employed as solid substance.

As evident from the foregoing discussion, various methodologies disclosed above claim a paclitaxel formulation with better stability. However, it is also clear from the above patents that all the efforts have been directed towards purification/treatment of cremophor by various means resulting in a stabilized paclitaxel formulation.

Some other patents also describe methods of stabilizing the paclitaxel formulation by presenting the components in kit form separating the drug from the degradant namely cremophor.

Ortner Peter in German Patent No. DE 19925211 discloses another approach wherein a kit for preparing stable paclitaxel formulation has been described. The kit comprises of three sealed vials, one containing separately stored drug (Paclitaxel), second containing solution of anhydrous citric acid in ethanol and the third containing Cremophor EL or ELP in ethanol. A method for preparing the formulation by mixing the contents of vials in a particular order has also been described. The author of the invention claims to provide a more economical alternative for providing stabilized paclitaxel composition by separate supply of active substance, sovent and stabilizer

Geczy Joszef et al in PCT international publication no. WO 98/57630 disclose another similar kit form for paclitaxel formulation. The invention concerns a pharmaceutical form for administering Paclitaxel, comprising two distinct containers one of which contains an ehanolic solution of Paclitaxel whereas another contains an ethanolic solution of Cremophor EL, Cremophor ELP or a mixture thereof. The contents of these two containers are mixed together and added to the perfusion liquid at the time of injecting to the patient. In the present disclosure authors have claimed a simple design which does not require any additional treatment of Cremophor.

Unfortunately, cremophor has certain serious side effects like hypersensitivity reactions requiring pre-medication with steroids and antihistaminic drugs. The cremophor formulation of paclitaxet also has a major clinical problem that it follows non-linear pharmacokinetics. This non-linearity is totally attributed to the presence of cremophor. Therefore, there is a need for alternate cremophor-free formulations possibly having linear pharmacokinetics.

One such attempt towards a cremohor-free formulation has been disclosed by the authors of this invention in U.S. Pat. Nos. 6,365,191 and 6,322,817. These patents describe a polymeric micellar nanoparticle formulation of paclitaxel, which is cremophor free. The preferred composition as described in the U.S. Pat. No. 6,365,191 can also be presented in a kit form comprising of two vials, one containing solution of pacltiaxel in alcohol and the other containing an aqueous solution of the co-polymer along with an anionic surfactant and a buffering agent to adjust the pH. The contents of the two vials are mixed at the time of infusing to the patient.

It is the object of this invention to provide a stabilized pharmaceutical composition of Paclitaxel in a solvent like dehydrated alcohol. More particularly, the present invention relates to stabilized alcoholic paclitaxel that lends itself to a nanoparticle formulation presented in a kit form suitable for bedside reconstitution.

### SUMMARY OF THE INVENTION

The invention is directed to a solvent suitable for preparing stabilized injection compositions containing at least one pharmaceutical agent. Accordingly, it is a primary object of the invention to provide a method of preparing a treated solvent having a stabilizing effect on the composition. and a method of preparing stabilized pharmaceutical compositions using the treated solvent.

The stabilized pharmaceutical composition produced from the treated solvent of the invention has been shown to have a shelf life greater than the compositions produced from untreated solvent. The solvent of the invention is particularly suitable for use with pharmaceutical compounds that exhibit decomposition, which is catalyzed by the presence of ionic, metallic and oxidizing impurities. Of particular interest are the antineoplastic agents such as paclitaxel, teniposide, camptothecin and derivatives thereof.

The solvent of the invention essentially comprises an alcohol. The preferred solvent includes absolute or dehydrated alcohol such as that sold by Merck. The absolute alcohol is treated to reduce the ionic, metallic and oxidizing impurities to a sufficiently low concentration to minimize the decomposition of the pharmaceutical agent that is catalyzed by the presence of these impurities. The content of impurities of the absolute alcohol is lowered by either treating the absolute alcohol with a strongly basic anion exchanger for example, Amberlite IRA 400 sold by Rohm and Haas Company to remove the anionic as well as other impurities or by the addition of a stabilizing agent possessing the combined properties of an acidifying agent, a chelating agent as well as an antioxidant and particularly, an organic acid such as malic acid.

The content of impurities of the absolute alcohol is also lowered by treating with Clays as low cost absorbents suitable for removal of trace impurities including mattalic and ionic impurities. Treatment of absolute alcohol with clays like Montmorillonite K-10 (Obtained from Aldrich Chemical Co.) was found to be effective in lowering the impurities levels resulting in a better stability profile. Montmorillonite K-10 is an inexpensive non-toxic powder, which can be easily filtered from the reaction mixture and may be reused.

The advantages of the invention are also attained by producing a stabilized pharmaceutical composition comprising at least one antineoplastic compound and a solvent capable of solubilising the antineoplastic compound, the solvent comprising a solubilizing amount of an alcohol such as absolute alcohol having been pre-treated to have an impurities content sufficiently low to substantially prevent degradation of the antineoplastic compound.

Further advantages of the invention are attained by providing a method of stabilizing a pharmaceutical composition containing a pharmaceutical agent such as paclitaxel, teniposide, camptothecin and derivatives thereof, and a solvent containing pre-treated absolute ethanol

### DETAILED DESCRIPTION OF THE INVENTION

The present invention is primarily directed to a solvent suitable for producing a stabilized pharmaceutical composition and to a method of producing and stabilizing a pharmaceutical composition.

Our invention provides a pharmaceutically stable formulation of paclitaxel. The process consists of treating absolute alcohol for removing various impurities. The content of ionic, metallic and oxidizing impurities of the alcohol can be lowered by a number of methods. In a first embodiment of the invention, the alcohol is contacted with specified quantities of a strongly basic anion exchange resin for specified time. This reduces the ionic impurities present in the alcohol.

In an another embodiment, absolute alcohol is contacted with clays like Montmorillonite K-10 for a specified period of time to reduce the ionic, metallic and oxidative impurities by way of absorption or adsorption.

In an alternative embodiment of the invention, the alcohol is treated by the addition of an organic acid and not a mineral acid, the preferred organic acid being Malic acid in a stabilizing amount to reduce the ionic, metallic and oxidizing impurities content to a sufficiently low level to substantially prevent degradation of the pharmaceutical compound catalyzed by these impurities.

The acid may be added to the alcohol before or after admixing with the pharmaceutical compound. Mineral acids such as, for example, HCl, HBr, HF, HI, H₂SO₄ and HNO₃ are not used as they are found to cause higher degradation of the drug than in the absence of these acids. Alternatively, organic acids such as lactic acid, citric acid and more preferably Malic acid may be used.

Malic acid has this unique triple action property of being an antioxidant, a chelating agent and an acidifying agent (Ref: Handbook of Pharmaceutical Excipients, 3^{rd} ed, edited by Arthur H. Kibbe; Pharmaceutical Press, UK)

Malic Acid as Antioxidant:

Malic acid is an alpha hydroxy acid popularly used as antioxidant, chelating and acidulating agents in many formulations. The antioxidative effect arises due to their ability to scavenge free radicals formed during oxidation reactions. Even as they are effective in the role of anti-oxidant effect per se, they are known to enhance antioxidative effect of other compounds since they can chelate metal ions. Studies indicate that these compounds may show an affinity for certain metal ions, for eg., malic acid can sequester aluminium ions strongly.

Malic Acid as Chelating Agent:

Malic acid also form chelates with the trace metals present in the solvent. The tentative structure of the chelate can be shown as follows in which two carboxylate ion of the malic acid can possibly play role as bidentate ligand

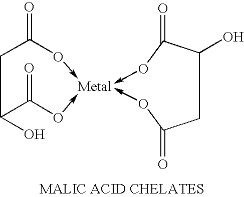

Malic Acid and Alcohol Improves Solvation:

The addition of alcohol and malic acid in the paclitaxel solution can enhance the stability due the solvation effect by forming intermolecular hydrogen bonding. The side chain of paclitaxel have —OH groups which can easily form hydrogen bonding with —COOH and —OH groups of malic acid and alcohol and further enhance the stability of the formulation.

The following non-limiting examples are intended to demonstrate the preferred embodiments of the invention. One skilled in the art will readily recognize that numerous embodiments of the invention can be practiced to achieve the stabilizing effect.

### EXAMPLE 1

This example was carried out to demonstrate the effect of pre-treatment of absolute alcohol with different types of ion exchange resins on the stability of Paclitaxel.

Sample 1a was prepared by dissolving 6 mg/ml of paclitaxel in absolute alcohol. The Sample 1b was prepared by pre-treating the absolute alcohol with a strongly basic anion exchange resin and then dissolving the paclitaxel in it to get a concentration of 6 mg/ml. The Sample 1c was prepared by pre-treating the absolute alcohol with a strongly acidic cation exchange resin and then dissolving the paclitaxel in it to get a concentration of 6 mg/ml. The sample 1d was prepared by pre-treating the absolute alcohol with a mixture of stongly acidic cation exchange resin and strongly basic anion exchange resin and then dissolving the paclitaxel in it to get a concentration of 6 mg/ml The samples were then subjected to an accelerated degradation study at 50° C. The results obtained are summarized as below:

TABLE 1

| | Degradation Products % at 50° C. | | | | | | | | | | | |
|---|---|---|---|---|---|---|---|---|---|---|---|---|
| | | | | | | | | | | | Total | |
| | | | | | | | | | | | (including | |
| | | | | | | | | | | | other | |
| | Baccatin | | EESC | | 10-DAP | | 10-DA-7-EP | | 7-EP | | degradation | |
| | 2 | 8 | 2 | 8 | 2 | 8 | | 8 | 2 | 8 | products) | |
| | Days | Days | Days | Days | Days | Days | 2 Days | Days | Days | Days | 2 Days | 8 Days |
| Sample 1a | 0.24 | 0.45 | 0.20 | 0.37 | 0.03 | 0.17 | 0.00 | 0.00 | 0.28 | 0.85 | 0.76 | 1.83 |
| Sample 1b | 0.03 | 0.21 | 0.02 | 0.17 | 0.05 | 0.07 | 0.00 | 0.00 | 0.17 | 0.39 | 0.26 | 0.84 |
| Sample 1c | 0.02 | 0.06 | 0.00 | 0.02 | 0.81 | 1.83 | 0.00 | 0.00 | 0.24 | 0.56 | 2.51 | 5.52 |
| Sample 1d | 0.02 | 0.06 | 0.00 | 0.02 | 0.34 | 1.03 | 0.00 | 0.00 | 0.14 | 0.38 | 0.50 | 2.30 |

EESC = Ethyl Ester Side Chain
10-DAP = 10 Deacetyl Paclitaxel
10-DA-7-EP = 10 Deacetyl 7 Epi-paclitaxel
7-EP = 7 Epi-paclitaxel

As shown in Table 1—Sample 1b prepared with absolute alcohol pre-treated with strongly basic anion exchange resin stabilizes paclitaxel maximum. Sample 1c prepared with absolute alcohol pre-treated with strongly acidic cation exchange resin increases the degradation of paclitaxel.

### EXAMPLE 2

This example was carried out to demonstrate the effect of addition of mineral acid versus organic acid to absolute alcohol on the stability of Paclitaxel.

A solution of Paclitaxel was prepared by dissolving 20 mg/ml of paclitaxel in absolute alcohol. The solution was divided into 6 parts in 6 different vials. Each of the samples 2a to 2f were mixed with the components listed in the table 2 so as that the pH was adjusted between 3.7 to 3.9. The samples were then subjected to an accelerated degradation study at 50° C. and then analysed by HPLC for degradation products. The results obtained are summarized as below:

TABLE 2

| | | | Percentage of Degradation Products | | | | | |
|---|---|---|---|---|---|---|---|---|
| | | | after 2 days at 50° C. | | | | | |
| | | | | | | | | Total |
| | | | | | | | | (including |
| | | pH (10% | | | | | | other |
| | Component | soln in | | | 10- | 10-DA-7- | 7- | degradation |
| | Added | water) | Baccatin | EESC | DAP | EP | EP | products) |
| Sample | None | 5.61 | 0.09 | 0.08 | 0.05 | 0.00 | 0.20 | 0.42 |
| 2a | | | | | | | | |
| Sample | HCl | 3.86 | 0.00 | 0.00 | 0.62 | 0.00 | 0.11 | 1.70 |
| 2b | (1%) | | | | | | | |
| Sample | Malic Acid | 3.78 | 0.00 | 0.00 | 0.00 | 0.00 | 0.11 | 0.11 |
| 2c | | | | | | | | |
| Sample | HCl + | 3.85 | 0.00 | 0.00 | 0.31 | 0.00 | 0.09 | 0.57 |
| 2d | Malic Acid | | | | | | | |
| Sample | Lactic Acid | 3.85 | 0.12 | 0.04 | 0.08 | 0.00 | 0.17 | 0.41 |
| 2e | | | | | | | | |
| Sample | Citric Acid | 3.80 | 0.00 | 0.00 | 0.02 | 0.00 | 0.15 | 0.17 |
| 2f | | | | | | | | |

EESC = Ethyl Ester Side Chain
10-DAP = 10 Deacetyl Paclitaxel
10-DA-7-EP = 10 Deacetyl 7 Epi-paclitaxel
7-EP = 7 Epi-paclitaxel

As shown in Table 2—Sample 2C stabilized with. Malic acid gives the maximum stability, followed by citric acid under stress condition of 50° C. Hydrochloric acid on the other hand increases the degradation of Paclitaxel in alcoholic solution

### EXAMPLE 3

After having shown the advantage of organic acids over mineral acids the following study was carried out for comparative evaluation of both the promising organic acids i.e. malic acid and citric acid under accelerated stability conditions.

A solution of Paclitaxel was prepared by dissolving 20 mg/ml of paclitaxel in absolute alcohol (Sample 3a). The solution was divided into 3 parts to make 3 types of samples comprising untreated (Sample 3b), 0.01% w/v Malic acid treated (Sample 3c) and 0.01% w/v Citric acid treated (Sample 3d) formulations. The samples were then subjected to an accelerated degradation study at 40° C. and then analyzed by HPLC for degradation products. The results obtained are summarized as below:

TABLE 3

| | | Percentage of Degradation Products | | | | | |
|---|---|---|---|---|---|---|---|
| | | | | | | | Total |
| | | | | | | | (including |
| | | | | | | | other |
| | Storage | | | 10- | 10-DA- | | degradation |
| | Condition | Baccatin | EESC | DAP | 7-EP | 7-EP | products) |
| Sample 3a | Initial | 0.01 | 0.01 | 0.00 | 0.00 | 0.06 | 0.08 |
| Sample 3b | 1 Month/40° C. | 0.41 | 0.33 | 0.05 | 0.00 | 0.25 | 1.03 |
| Control/ | 2 Month/40° C. | 0.39 | 0.33 | 0.08 | 0.00 | 0.31 | 1.11 |
| Untreated | 3 Month/40° C. | 0.41 | 0.33 | 0.11 | 0.04 | 0.33 | 1.22 |
| Sample 3c/ | 1 Month/40° C. | 0.09 | 0.00 | 0.03 | 0.00 | 0.09 | 0.21 |
| Malic acid | 2 Month/40° C. | 0.10 | 0.00 | 0.04 | 0.00 | 0.11 | 0.25 |
| treated | 3 Month/40° C. | 0.10 | 0.00 | 0.06 | 0.00 | 0.11 | 0.27 |
| Sample 3d/ | 1 Month/40° C. | 0.09 | 0.00 | 0.02 | 0.00 | 0.09 | 0.20 |
| Citric acid | 2 Month/40° C. | 0.11 | 0.00 | 0.04 | 0.00 | 0.14 | 0.29 |
| treated | 3 Month/40° C. | 0.10 | 0.00 | 0.06 | 0.00 | 0.11 | 0.28 |

EESC = Ethyl Ester Side Chain
10-DAP = 10 Deacetyl Paclitaxel
10-DA-7-EP = 10 Deacetyl 7 Epi-paclitaxel
7-EP = 7 Epi-paclitaxel

As shown in Table 3—Sample 3c and Sample 3d stabilized with Malic acid and Citric acid respectively result in better stability as compared to untreated sample 3b.

### EXAMPLE 4

This example was carried out to demonstrate the effect of clay treatment of absolute alcohol on the stability of Paclitaxel.

In the study 10 g of Montmorillonite K-10 (Obtained from Aldrich Chemical Co.) was added to 100 ml of absolute Alcohol (E-Merck) at 25-30° C. and the mixture was stirred mechanically for 10 hrs. At the end of 10 Hrs. the mixture was filtered through 1μ Nylon filter paper. A solution of Paclitaxel was prepared by dissolving 20 mg/ml of paclitaxel in treated absolute alcohol (sample 4a) and its stability was compared against a control solution prepared in untreated absolute alcohol (sample 4b). Both the samples 4a & 4b were subjected to an accelerated degradation study at 50° C. and then analyzed by HPLC for degradation products. The results obtained are summarized as below:

TABLE 4

| | | Percentage of Degradation Products at 50° C. | | | | | |
|---|---|---|---|---|---|---|---|
| | | | | | | | Total |
| | | | | | 10- | | (including other |
| | | | | 10- | DA- | | degradation |
| | Time | Baccatin | EESC | DAP | 7-EP | 7-EP | products) |
| Sample 4a | 2 Days | 0.15 | 0.12 | 0.16 | 0.00 | 0.40 | 0.83 |
| (Untreated) | 8 Days | 0.48 | 0.36 | 0.24 | 0.00 | 0.68 | 1.76 |
| Sample 4b (Clay | 2 Days | 0.00 | 0.00 | 0.00 | 0.00 | 0.12 | 0.12 |
| Treated) | 8 Days | 0.01 | 0.00 | 0.03 | 0.00 | 0.14 | 0.18 |

EESC = Ethyl Ester Side Chain
10-DAP = 10 Deacetyl Paclitaxel
10-DA-7-EP = 10 Deacetyl 7 Epi-paclitaxel
7-EP = 7 Epi-paclitaxel

As shown in Table 4—Sample 4b prepared with Clay treated absolute alcohol shows better stability as compared to the corresponding control sample prepared with untreated solvent.

### EXAMPLE 5

This example demonstrates a kit presentation for bedside reconstitution of the Paclitaxel nanoparticle formulation. The presentation comprises two distinct containers, ampoules, a dual chamber syringe, vials etc., one of which contains paclitaxel solution in a solvent preferably dehydrated alcohol. The dehydrated alcohol may optionally be pre-treated as in examples 1 to 4 so as to have a stabilizing action on the stability of paclitaxel in the composition. The other container, ampoule, chamber, vial contains a solubilizer that can be a concentrate/combination of excipients that can help keep the drug in colloidal solution when mixed together with a clinically acceptable aqueous dilution fluid for infusion. The kit presentation also covers the combination of excipients suitable for forming nanoparticles as disclosed in the U.S. Pat. No. 6,365,191, especially the preferred composition as covered in the claim 12 of the U.S. Pat. No. 6,365,191 by the authors of this invention.

It may be noted that the containers such as vials, ampoules, dual chamber syringes etc are just mentioned as example and should not be taken as limiting examples. The basic essence is to keep the two solutions i.e. the drug solution in the solvent and the solubilizer separately till use so as to not only minimize the loss of potency of drug during shelf-life of the product but also to enable bedside reconstitution of nanoparticle formulation.

### EXAMPLE 6

This examples demonstrates the importance of the method of bedside reconstitution of the Paclitaxel colloidal formulation, more particularly with reference to the nanoparticle formulation as disclosed in the U.S. Pat. No. 6,365,191 by the authors of this invention (incorpoarted herein I entirety by refernce). It was observed that that method of addition of the ethanolic solution of paclitaxel into the dilution fluid (10% dextrose) containing combination of excipients as mentioned in the preferred formulation in the U.S. Pat. No. 6,365,191 has a major bearing on the size of the nanoparticles formed and thus on the physical stability of the reconstituted nanoparticle solution. The dehydrated alcohol used herein may optionally be treated as shown in example 1 to 4. Several nanoparticle formulations were prepared at different drug concentration levels varying from 0.1 to 1.6 mg/ml. The ratio of drug to polymer was kept constant (2:1) in the final nanoparticle formulation. Infusion vehicle was obtained by mixing requisite amounts of the excipients containing 10 mg/ml of polymer, 10 mg/ml of Tri-sodium citrate and 6.66 mg/ml of sodium deoxycholate with 10% dextrose solution resulting in a solution of pH range 6.5 to 7.1. Requisite amount of alcoholic Paclitaxel (20 mg/ml) solution was taken in a disposable syringe fitted with a 29G, ½″ needle and then added to reconstituted infusion fluid contained in a 50 ml Polystyrene centrifuge tube. Ethanolic Paclitaxel was added in two different ways. Slow injection was given by slowly adding Ethanolic Paclitaxel drop by drop whereas rapid injection was given ensuring that the content of the syringe flow out in a continuous rapid stream in one go without any interruption. The position of the needle was kept vertically in the middle just below the upper surface of the infusion fluid. Care was taken to exclude any air bubble left in the syringe prior to the injection. Results are shown below in table-6

TABLE 6

| | Concen- | | | | |
|---|---|---|---|---|---|
| | tration | | | | |
| | of Drug | Drug Addition | Size of Nanoparticles | | |
| | (mg/ml) | Technique | Initial | 24 Hrs. | Stability |
| Sample 7a | 0.4 | Slow Injection | >250 nm | Precipitation | <5 min |
| | | | | observed | |
| Sample 7b | 0.4 | Rapid Injection | 75.8 nm | 84.8 nm | >24 Hrs |
| Sample 7c | 0.6 | Slow Injection | >250 nm | Precipitation | <5 min |
| | | | | observed | |
| Sample 7d | 0.6 | Rapid Injection | 76.2 nm | 83.6 nm | >24 Hrs |
| Sample 7e | 0.8 | Slow Injection | >250 nm | Precipitation | <5 min |
| | | | | observed | |
| Sample 7f | 0.8 | Rapid Injection | 84.8 nm | 88.5 nm | >24 Hrs |
| Sample 7g | 1.0 | Slow Injection | >250 nm | Precipitation | <5 min |
| | | | | observed | |
| Sample 7h | 1.0 | Rapid Injection | 85.8 nm | 94.9 nm | >24 Hrs |

As evident from table-6, rapid injection technique results in much better stability and particle size less than 100 nm which is very important for nanoparticle based drug delivery systems

It should be clearly understood that the rapid injection technique in its general aspect is not limited to the specific details referred to herein above. The basic concept is based on “rapid injection” of alcohlic solution of hydrophobic drug in aqueous solution of amphiphilic polymer or coploymer capable of forming polymeric micelles, resulting in nanoparticle formation.

What is claimed is:

1. A stabilized pharmaceutical composition comprising an antineoplastic compound and a solvent capable of solubilizing said antineoplastic compound comprising alcohol that has been stabilized by the addition/treatment of/with a stabilizing agent.

2. A composition as claimed in claim 1 wherein said stabilizing agent is an acid.

3. A composition as claimed in claim 2 wherein said stabilizing agent is a non mineral acid.

4. A composition as claimed in claim 3 wherein any metallic, ionic and oxidative impurities present therein has been removed/reduced by complexation or neutralization.

5. A composition as claimed in claim 3 wherein said antineoplastic compound is selected from the group consisting of paclitaxel, teniposide, camptothecin and derivatives thereof.

6. A composition as claimed in claim 3 wherein said alcohol is absolute alcohol.

7. A composition as claimed in claim 4, wherein said alcohol has been pre-treated to reduce the metallic, ionic and oxidative impurities by contacting with an ion-exchange resin.

8. A composition as claimed in claim 7 wherein said ion-exchange resin is a strongly-basic anion exchange resin.

9. A composition as claimed in claim 7 wherein said ion exchange resin is in the form of beads and wherein one litre of alcohol is contacted with 50 to 800 gm more preferably 200 to 600 gm of the ion-exchange resin.

10. A composition as claimed in claim 3 wherein said acid possesses unique triple action properties of an antioxidant, a chelating agent and an acidifying agent.

11. A composition as claimed in claim 10 wherein said acid is malic acid or citric acid

12. A composition as claimed in claim 1 wherein said alcohol has been pre-treated to reduce the metallic, ionic and oxidative impurities by contacting with clay.

13. A composition as claimed in claim 12 wherein said clay is Montmorrilonite K-10.

14. A composition as claimed in claim 12 wherein 20-200 ml of alcohol is contacted with 2-20 gm of clay at 10-80° C., the mixture is then stirred mechanically for 2-30 hrs and filtered through 1μ Nylon filter.

15. A composition as claimed in claim 14 wherein about 80-120 ml of alcohol is contacted with 8- 10 gm of clay at 25-30° C. for 8-12 hrs. and the treated alcohol thus obtained is used as such for the formulation.

16. A stabilized pharmaceutical composition comprising an antineoplastic compound selected from the group consisting of teniposide, paclitaxel, camptothecin, and derivatives thereof and a solvent comprising an alcohol that has been stabilized by the addition of malic acid or citric acid.

17. A method for the preparation of a stabilized pharmaceutical composition which comprises adding to an antineoplastic compound, a solvent comprising of an alcohol that has been stabilized by the addition/treatment of/with a stabilizing agent.

18. A method as claimed in claim 16 wherein said said stabilizing agent is an acid.

19. A method as claimed in claim 16 wherein said stabilizing agent is a non mineral acid.

20. A method as claimed in claim 16 wherein any metallic, ionic and oxidative impurities present therein has been removed/reduced by complexation or neutralization.

21. A method as claimed in claim 16 wherein said antineoplastic compound is selected from the group consisting of paclitaxel, teniposide, camptothecin and derivatives thereof.

22. A method as claimed in claim 16 wherein said alcohol is absolute alcohol.

23. A method as claimed in claim 16 wherein said alcohol has been pre-treated to reduce the metallic, ionic and oxidative impurities by contacting with an ion-exchange resin.

24. A method as claimed in claim 22 wherein said ion-exchange resin is a strongly-basic anion exchange resin.

25. A method as claimed in claim 22 wherein said ion exchange resin is in the form of beads and wherein one litre of alcohol is contacted with 50 to 800 gm more preferably 200 to 600 gm of the ion-exchange resin.

26. A method as claimed in claim 18 wherein said acid possesses a unique triple action properties of an antioxidant, a chelating agent and an acidifying agent.

27. A method as claimed in claim 25 wherein said acid is malic acid or citric acid

28. A kit suitable for bedside reconstitution comprising two distinct containers, ampoules, a dual chamber syringe, or vials one of which contains paclitaxel solution in a solvent consisting of a dehydrated alcohol that has been, optionally pre-treated to reduce metallic, ionic and oxidative impurities by complexation, absorption, removal or neutralization of impurities or stabilized by the addition of a stabilizing agent and the other container, ampoule, chamber, vial containing a solubilizer such as a concentrate/combination of excipients for keeping the drug in solution when mixed together with a clinically acceptable aqueous dilution fluid for infusion.

29. A kit as claimed in claim 27 wherein said combination of excipients comprises an amphiphilic polymer capable of forming micellar nanoparticles alongwith suitable anionic surfactant and a buffering agent.

30. A kit as claimed in claim 27 wherein said alcohol has been pretreated with an acid.

31. A kit as claimed in claim 27 wherein said acid is a non mineral acid.

32. A kit as claimed in claim 27 wherein said acid is malic acid or citric acid.

33. A kit as claimed in claim 27 wherein said alcohol has been pretreated with an ion exchange resin

34. A kit as claimed in claim 27 wherein said alcohol has been pretreated with a clay like Montmorrilonite K-10
